(19) United States
(12) Patent Application Publication
(10) Pub. No.: US 2020/0069861 A1
(43) Pub. Date: Mar. 5, 2020

(54) APHERESIS TO REDUCE HIGH BLOOD PRESSURE IN PRE-ECLAMPSIA

(71) Applicant: Henry J. Smith, Temecula, CA (US)

(72) Inventor: Henry J. Smith, Temecula, CA (US)

(21) Appl. No.: 16/432,771

(22) Filed: Jun. 5, 2019

Related U.S. Application Data
(62) Division of application No. 62/763,472, filed on Jun. 18, 2018.

(51) Int. Cl. A61M 1/36; A61M 1/34

(52) U.S. Cl. CPC A61M 1/362; A61M 1/3496

(57) ABSTRACT

There are certain factors in the blood of pregnant women with pre-eclampsia that appear to be associated with the disease. These include a soluble variant of the fms-like tyrosine kinase receptor (sFlt-1), soluble Endoglin (sEndoglin), and Endothelin-1. There is also evidence that hypertension may be caused by Na/K ATPase inhibitors such as digitalis-like factor, ouabain-like factors, marinobufogenin and marinobufotoxin. This invention teaches the removal of multiple harmful factors using a combination of targeted apheresis and dialysis and/or ultrafiltration. Harmful factors that are proteins are bound out using immobilized binding agents such as antibodies, aptamers and binding peptides, while small molecule harmful factors are dialyzed out or filtered out. Removal of multiple harmful factors is expected to ameliorate the symptoms of pre-eclampsia and prolong pregnancy.

### CROSS-REFERENCE TO RELATED APPLICATIONS

This application claims priority to provisional application U.S. 62/763,472 titled “Apheresis to reduce high blood pressure” and filed Jun. 18, 2018.

### STATEMENT RE: FEDERALLY SPONSORED RESEARCH/DEVELOPMENT

Not Applicable

### BACKGROUND INFORMATION

Pre-eclampsia or toxemia during pregnancy is one of the leading causes of maternal and infant mortality. The symptoms of pre-eclampsia typically appear after the 20th week of pregnancy and are characterized by high blood pressure, edema and protein in the urine. In severe cases there is a massive rise in blood pressure that can result in severe complications such as cardiac failure or stroke, and sometimes death of the mother or baby.

Pre-eclampsia can vary in severity from mild to life threatening. The mild form of pre-eclampsia is usually treated with bed rest and frequent monitoring. For moderate to severe cases, hospitalization is recommended and the patient is treated with blood pressure medication or anticonvulsant medications to prevent seizures. Unfortunately there is currently no really effective treatment for pre-eclampsia and if the condition worsens and becomes life-threatening the baby is delivered prematurely.

Babies born very pre-term have a high risk of dying or being physically or mentally disadvantaged for life. It would be desirable to develop a safe and effective means of prolonging pregnancy for as long as possible so that a healthier baby is born at term or as close to term as possible.

There are intensive ongoing research studies on identifying the causative factors for pre-eclampsia in the hope that these may lead to improved therapies for this condition. There appears to be numerous factors that may be implicated including; maternal immunologic intolerance, abnormal placental implantation, genetic, nutritional and environmental factors, and cardiovascular and inflammatory changes. For example, there are elevated levels of certain factors in the blood of pregnant women with pre-eclampsia that appear to be associated with the disease. In particular there is a soluble variant of the fms-like tyrosine kinase receptor called sFlt-1 (aka soluble vascular endothelial growth factor receptor1 or sVEGFR1) that appears to disrupt normal placental development by binding to certain growth hormones called vascular endothelial growth factor (VEGF) and Placental Growth Factor (PIGF) and preventing them from stimulating normal placental development. Another circulating protein that appears to play an important role in pre-eclampsia is soluble Endoglin (s-Endoglin). S-Endoglin can bind to Transforming Growth Factor-beta (TGF-beta) and prevent it from binding to endothelial cells and thus impair their capacity for forming blood capillaries. Yet another circulating factor that appears to be associated with pre-eclampsia is Endothelin-1. Endothelins are peptides produced in the endothelium that function to constrict blood vessels and raise blood pressure. They are normally kept in balance by other mechanisms, but when they are over-expressed, they contribute to high blood pressure and heart disease.

There is also growing evidence that hypertension may be caused by an endogenous factor that inhibits the Na/K ATPase enzyme of vascular smooth muscle cells and triggers vasoconstriction resulting in high blood pressure. Although the factor responsible for inhibiting Na/K ATPase and causing hypertension has not been definitely confirmed in humans there are various studies that have identified several promising candidates including an endogenous digitalis-like factor, ouabain or ouabain-like factor, marinobufogenin (MGB), and marinobufotoxin (MBT). It is likely that more Na/K ATPase inhibitors will be discovered over time and also that different forms of hypertension may be triggered by one or more different Na/K ATPase inhibitors.

Other factors that appear to be implicated in pre-eclampsia are pro-inflammatory cytokines such as Tumor Necrosis Factor-alpha (TNF-alpha), Interleukin-1 (IL-1), interleukin-6 (IL-6) and interleukin-8 (IL-8) that could contribute to pathogenesis of the disease.

There appears to be multiple circulating factors associated with pre-eclampsia. It is possible that additional factors will be discovered in the future and that more than a single factor will be involved in the pathogenesis of pre-eclampsia. Therefore removal of as many of these contributing factors as possible is expected to alleviate the symptoms of the disease.

This invention teaches a novel method of treating pre-eclampsia by removing multiple circulating harmful factors from the blood of the patient with pre-eclampsia using a combination of targeted apheresis and dialysis and/or ultrafiltration.

The art is silent of the use of targeted apheresis combined with dialysis and/or ultrafiltration as a means of treating high blood pressure and other symptoms associated with pre-eclampsia.

### BRIEF SUMMARY

There are certain factors in the blood of pregnant women with pre-eclampsia that appear to be associated with the disease. This invention teaches the removal of multiple harmful factors using a combination of targeted apheresis and dialysis and/or ultrafiltration. Harmful factors that are proteins are targeted and bound out using immobilized binding agents, while small molecule harmful factors are dialyzed out and/or filtered out. Removal of multiple harmful factors is expected to ameliorate the symptoms of pre-eclampsia and prolong pregnancy so that the baby is born as close to term as possible.

### DESCRIPTION OF INVENTION

This invention teaches a novel combination of targeted apheresis and dialysis and/or ultrafiltration to remove multiple circulating harmful factors in the blood associated with causing high blood pressure. Briefly, small molecule harmful factors are removed using dialysis or ultrafiltration while large molecule harmful factors are removed using targeted apheresis. The protocol described herein is basically similar to the methods used in dialysis and conventional therapeutic apheresis. These established protocols are described in detail by many manufacturers and suppliers of dialyzers and apheresis equipment and are well-known to those of skill in the art.

This invention however, teaches certain embodiments that are not disclosed in the prior art. For example, renal dialysis treats whole blood while conventional therapeutic apheresis treats plasma. In this invention whole blood is treated using targeted apheresis and dialysis and/or ultrafiltration. Further, therapeutic apheresis often uses an arterio-venous shunt while renal dialysis uses a veno-venous shunt. In this invention although both access methods can be used the veno-venous shunt is preferred because it is less traumatic to the patient, and the period of apheresis treatment can be performed over an extended period of time. In general a longer duration of apheresis is preferable as it allows a higher removal of harmful factors in a gradual fashion which in turn reduces risk of an adverse reaction in the patient. The method of targeted apheresis disclosed in this invention also differs from conventional therapeutic apheresis in that in targeted apheresis immobilized binding agents such as antibodies, aptamers or binding peptides are used to specifically target and bind out harmful factors in the blood.

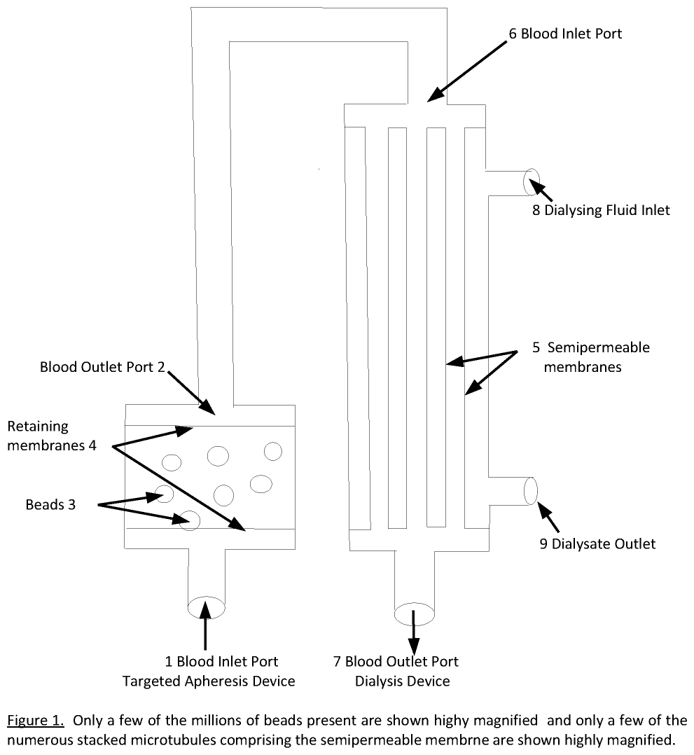

In one embodiment of this invention two devices consisting of a targeted apheresis device and a dialysis device arranged in sequence are used. The targeted apheresis device can be positioned either before or after the dialysis device. Typically, the targeted apheresis device contains one or more binding agents immobilized on a supporting structure that may be in the form of beads, or plastic sheets, or membranes. The targeted apheresis device is connected to a renal dialysis device containing a semipermeable membrane that will permit thru passage of water and small molecules while retaining large molecules that exceed the exclusion limit of the pores of the membrane. Typically the exclusion limit of the membrane is about MW 30,000 but can vary according to different manufacturers. Some examples of the two device configurations are illustrated in FIGS. 1-3.

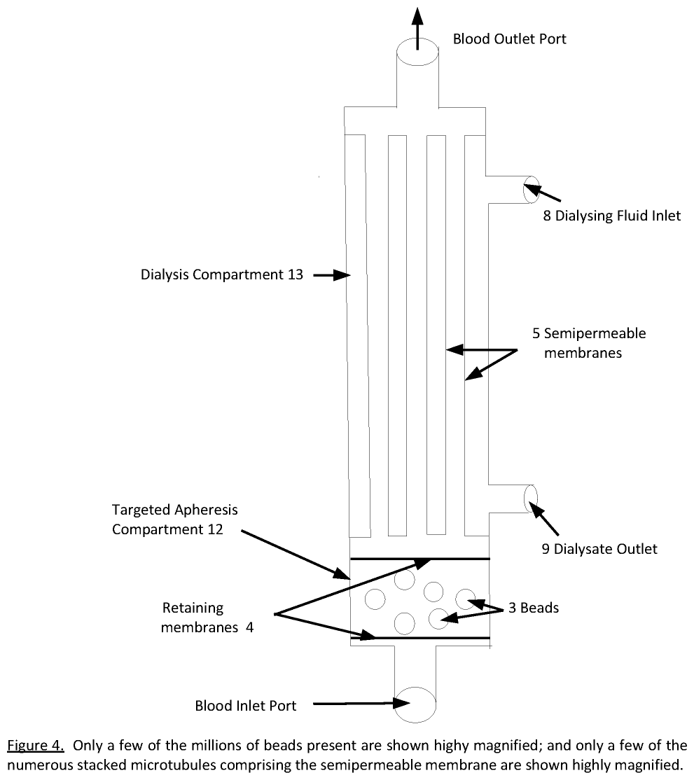

In one embodiment of this invention the targeted apheresis device and the dialysis device are combined into a single device with two compartments. One compartment is used to perform targeted apheresis and the other compartment to perform dialysis as illustrated in FIG. 4. The targeted apheresis chamber will contain the immobilized binding agents attached to any one of a variety of different support matrixes. For example, as described earlier, these may be in the form of beads, or sheets, or support membranes. The process of targeted apheresis combined with dialysis using the single device is essentially the same as that disclosed when two devices are used.

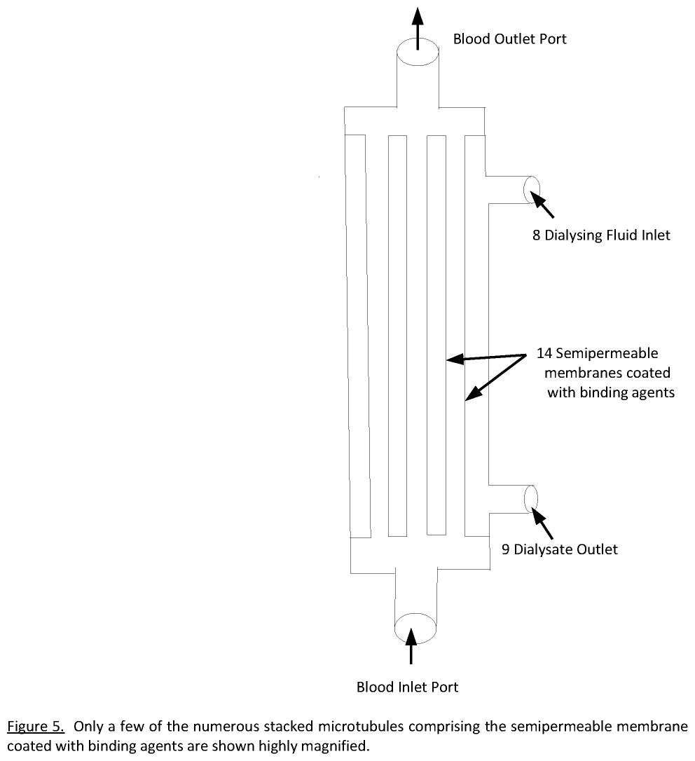

In one embodiment of this invention there is a single device that simultaneously performs targeted apheresis and dialysis using a semipermeable membrane coated with one or more binding agents as illustrated in FIG. 5.

In this invention the term “harmful factor” or “factor” is a general all-inclusive term used to describe all the harmful factors in the blood that are either directly or indirectly implicated in causing high blood pressure. This includes sFlt-1, sEndoglin, Endothelin-1, digoxin, endogenous digitalis-like factor (EDLF), endogenous ouabain or ouabain-like factor, marinobufogenin (MGB), marinobufotoxin (MBT), tumor necrosis factor (TNF), interleukin-1 (IL-1), interleukin 6 (IL-6) and interleukin 8 (IL-8).

In this invention the term “inhibitor” will refer to the Na/K ATPase inhibitor and will include all the known Na/K ATPase inhibitors including digoxin and digitalis-like factor, ouabain and ouabain-like factor, marinobufogenin, and marinobufotoxin.

In this invention the term “binding agent” will refer to antibodies, aptamers and binding peptides that target circulating harmful factors, and will include all of the varieties within each category of binding agents. For example, the term antibody will include polyclonal, monoclonal and recombinant antibodies as well as the binding fragments of those antibodies, The term “aptamer” will include all types and varieties of aptamers including single-strand DNA, single strand RNA, branched aptamers and chemically modified aptamers. The term “binding peptide” will include the whole peptide molecule or the active site on the peptide molecule capable of binding to its target.

### Targeted Apheresis and Dialysis to Treat Pre-Eclampsia

The following examples will illustrate the general principles of this invention. One of skill in the art will recognize that there are many modifications and variations that can be made without departing from the spirit and scope of this invention. These examples are therefore not to be construed as limiting but used instead to illustrate the versatility and broad scope of this invention as a means of treating hypertension using apheresis

### Example 1

In one embodiment of this invention a targeted apheresis device is connected to a dialysis device as illustrated in FIG. 1. The targeted apheresis device consists of a container filled with one or more binding agents attached to beads (3). The binding agent is typically an antibody against a particular harmful factor, and the beads are typically made of highly porous cross-linked agarose. For example, a harmful factor associated with pre-eclampsia is sFlt-1 and the binding agent to remove sFlt-1 is an anti-sFlt-1 antibody attached to cross-linked agarose beads. The beads are enclosed within a container with an inlet port (1) to allow entry of blood and an outlet port for the blood to exit (2). The beads (3) are retained within the container by a highly porous membrane or sieve (4) at the top and another at the bottom of the device. The beads are typically manufactured to be larger than 100 um in diameter and permeated throughout with large pores capable of allowing large molecules to penetrate into the interior of the bead. The interstices of the retaining mesh or sieves are manufactured to be large enough (e.g. 50-100 um pores) to allow blood to freely pass thru them yet small enough to retain the beads. In order to permit unimpeded blood flow thru the targeted apheresis device it is important to ensure that there is a large filtering surface area for the top and bottom retaining mesh or sieve.

As the blood passes thru the targeted apheresis device one or more harmful factors in the plasma fraction of the blood will come into contact with the binding agents coating the exterior of the beads and also coating the interior pores of the beads. The large pores in the beads will allow the harmful factors in the plasma to bind to the immobilized binding agents coating the exterior surfaces and interior pores of the beads. The treated blood then passes through an inlet port (6) into a dialysis device consisting of a chamber with a semipermeable membrane (5) having pores capable of dialyzing out small molecules while retaining blood cells and large molecules such as plasma proteins. Typically the exclusion limit for the dialysis membrane is approximately MW 30,000 with some variation between manufacturers. In this invention it is recommended that the exclusion limit of the membrane not exceed MW 30,000 in order that beneficial components such as hormones (e.g. VEGF and PIGF) are retained in the blood. There is an inlet port for blood to enter the device (6) and an outlet port for the blood to exit (7). There is also an entry port (8) to allow water or dialyzing fluid to enter the device and an exit port (9) to allow the dialysate to be removed. As the Na/K ATPase inhibitors have MW less than the exclusion limit of the semipermeable membrane they will pass thru the semipermeable membrane and removed with the dialysate.

It will be obvious to one of skill in the art that from this basic teaching there are many other binding agents and support matrixes that can be employed. For example, other binding agents such as binding peptides and aptamers that mimic the binding of an antibody to its antigen can be used in lieu of antibody in the targeted apheresis device. The methods of attaching the binding agent to solid surfaces are also known to those of skill in the art and will be described in more detail later.

One of skill in the art will also recognize that there are other support matrixes that can be used in lieu of agarose beads. For example, there are a variety of beads made of different materials that can be used including silica beads, acrylic beads, polystyrene bead, latex beads and similar materials can be used. The utilization of a variety of beads of different sizes and composition is therefore considered to lie within the spirit and scope of this invention.

### Example 2

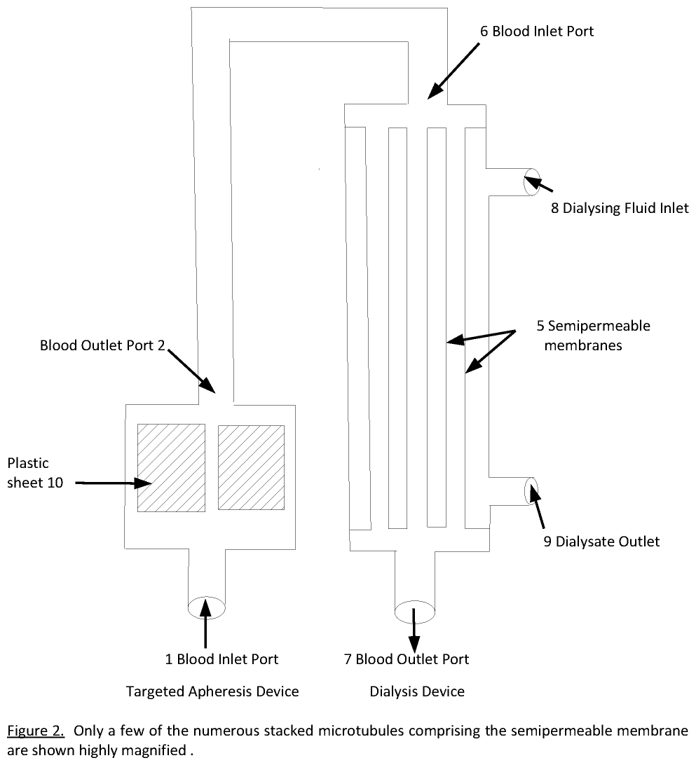

In one embodiment of this invention a targeted apheresis device is connected to a dialysis device as illustrated in FIG. 2. Within the targeted apheresis device there are one or more plastic sheets (10) coated with one or more binding agents. In order to further increase the surface area for attachment of the binding agent the plastic sheets are manufactured to have a coarse surface. For example, the sheets can be manufactured to have densely packed microgrooves or micropillars. The plastic sheets being larger than the inlet and outlet ports will be retained within the device without requiring retaining membranes. There is an inlet port (1) for blood to enter the apheresis device and an outlet port (2) for the blood to exit. When blood is pumped thru the device the immobilized binding agent on the sheets will bind out the harmful factor and the blood then passes through an inlet port (6) into the dialysis device. The dialysis device contains a semi-permeable membrane (5) where small molecule harmful factors are dialyzed out before the blood exits through an outlet port (7) and is returned to the patient. There is an inlet port (8) for dialysing fluid to enter the chamber and an outlet port (9) for removal of the dialysate and small molecule harmful factors.

### Example 3

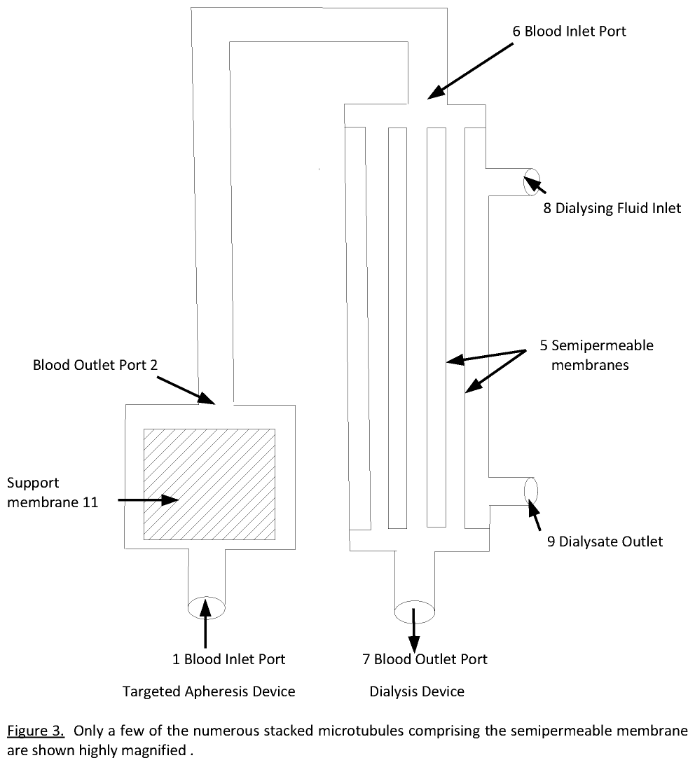

In one embodiment of this invention a targeted apheresis device is connected to a dialysis device as illustrated in FIG. 3. Within the targeted apheresis device is a membrane (11) which is utilized as the support matrix for one or more binding agents. The primary purpose of the membrane is to provide a large surface area for attaching the binding agent. In the preferred embodiment the membrane is an apheresis membrane that has a large surface area especially when the internal surface area of the pores of the membrane is taken into consideration. The membrane may be in the form of a flat or pleated sheet or composed of microtubules embedded within the membrane. In this invention the type of membrane typically used in therapeutic apheresis is preferred. There are a variety of membranes made of different materials including cellulose, cellulose acetate, polysulfone, polyacrylonitrile, polyimide, polyethylene, polymethyl methacrylate, ethylene-vinyl alcohol copolymer and others. Membranes may also be composed of combinations of different materials. There is an inlet port (1) for blood to enter the apheresis device and an outlet port (2) for the blood to exit. When blood is pumped thru the device the immobilized binding agent on the membrane will bind out the harmful factor and the blood then passes through an inlet port (6) into the dialysis device. The dialysis device contains a semi-permeable membrane (5) where small molecule harmful factors are dialyzed out before the blood exits through an outlet port (7) and is returned to the patient. There is an inlet port (8) for dialysing fluid to enter the chamber and an outlet port (9) for removal of the dialysate and small molecule harmful factors.

### Example 4

In one embodiment of this invention the targeted apheresis device and the dialysis device are combined into a single device as illustrated in FIG. 4. The device is divided into several compartments that perform different functions. For example, one compartment (12) will perform the targeted apheresis function and another compartment (13) will perform the dialysis function. The targeted apheresis compartment (12) will contain one or more immobilized binding agents coated on a support matrix (3) e.g. beads, or plastic sheets or membranes. Briefly, blood is pumped thru through the blood inlet port into the targeted apheresis compartment containing one or more immobilized binding agents (e.g. beads coated with the binding agent). The harmful factor is bound out and the blood then passes into the dialysis compartment (13). The dialysis compartment contains a semi-permeable membrane (5) where small molecule harmful factors are dialyzed out before the blood exits through the blood outlet port and is returned to the patient. There is an inlet port (8) for dialysing fluid to enter the chamber and an outlet port (9) for removal of the dialysate and small molecule harmful factors.

### Example 5

In one embodiment of this invention a single apheresis device that can simultaneously perform targeted apheresis and dialysis is illustrated in FIG. 5. There is an blood inlet port for the blood to enter the device and a blood outlet port for the blood to exit This single apheresis device has a semipermeable membrane coated with one or more binding agents (14) that can target and bind out circulating harmful factors as the blood is pumped thru the device. At the same time that the binding agents on the semi-permeable membrane are binding out large molecule harmful factors the semipermeable membrane is exposed to a dialysing solution. Small molecule harmful factors will pass thru the semipermeable membrane into the dialysing solution. There is an inlet port (8) for dialysing fluid to enter the chamber and an outlet port (9) for removal of the dialysate and small molecule harmful factors. The cleaned blood is returned to the patient.

### Binding Agents

The binding agents generally fall into three classes—antibodies, binding peptides and aptamers. For example, if the harmful factor was sFlt-1 then the binding agent would be either an anti-sFlt-1 antibody or an anti-sFlt-1 binding peptide or an anti-sFlt-1 aptamer. Similarly, if the harmful factor was sEndoglin then the binding agent would be either an anti-sEndoglin antibody, or an anti-sEndoglin binding peptide or an anti-sEndoglin aptamer. The same principles would apply to the binding agents used to target each of the other harmful factors.

### Antibody.

In this invention the term “antibody” is used to include polyclonal antibodies, monoclonal antibodies and recombinant antibodies; and also the binding fragments (i.e. Fab) of these antibodies.

In one embodiment of this invention polyclonal antibodies are prepared by immunizing various species of animals against the factor. Typically, the animals used are rabbits, goats, sheep and horses but other animals can also be used. The antisera from immunized animals are treated to isolate and purify the antibodies using established methods including salt-fractionation, gel-filtration and affinity chromatography. These and other methods of purifying antibodies are known to those of skill in the art.

In one embodiment of this invention monoclonal antibodies are produced against the factor. The methods of producing monoclonal antibodies are known to those of skill in the art. Typically, monoclonal antibodies are produced using murine hybridoma technology. In order to avoid exposing the patient to foreign proteins (e.g. murine antibodies) the monoclonal antibodies are often “humanized” by replacing certain portions of the mouse antibody protein with human material. The monoclonal antibodies can be purified using standard down-stream processing techniques such as affinity binding to Protein A. These and other methods of developing and purifying monoclonal antibodies are known to those skilled in the art and are within the scope of this invention

In one embodiment of this invention recombinant antibodies are produced using genetic engineering technology. Typically a wide variety of antibody binding domains are expressed as membrane surface or viral coat proteins (phage display). The antibody binding domain that binds to the desired target (antigen) is then isolated along with the corresponding genes which can be sequenced and introduced into various expression hosts (e.g. bacterial, yeast or mammalian cells) and used to produce a high yield of antibodies. The recombinant antibodies can be purified using standard down-stream processing techniques such as His-tagging the antibodies and isolating them using immobilized metal affinity chromatography. These and other methods of developing and purifying recombinant antibodies are known to those skilled in the art and are within the scope of this invention

### Binding Peptide.

A binding peptide is comprised of a chain of aminoacids that are synthesized and selected to target the harmful factor. There are various methods for preparing synthetic or biological peptide libraries composed of up to a billion different sequences, and for identifying a particular peptide sequence that will target a particular epitope. Typically a large number of different peptide sequences are allowed to react with the target and the peptide with the highest binding affinity is isolated and sequenced. Once the binding peptide sequence is identified increased quantities of that binding peptide can be produced by synthesis or using genetic engineering technology. The means of producing synthetic or biologically derived peptides are known to those of skill in the art and are within the scope of this invention.

### Aptamer.

An aptamer is a single stranded DNA or single stranded RNA molecule that is synthesized to specifically bind to its target. Aptamers are small (i.e. 40 to 100 bases), synthetic oligonucleotides. They may be composed as a single-stranded DNA chain (ssDNA) or a single-stranded RNA chain (ssRNA). Each aptamer has a unique configuration as a result of the composition of the nucleotide bases in the chain causing the molecule to fold in a particular manner. Because of their folded structure each aptamer will bind selectively to a particular epitope in a manner analogous to an antibody binding to its antigen. In order to improve bioavailability against nucleases found in vivo the oligonucleotides comprising the aptamer may be modified to avoid nuclease attack. They may for example be synthesized as L-nucleotides instead of D-nucleotides and thus avoid degradation from nucleases present in blood.

Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand. Once the nucleotide sequence is identified increased quantities of that aptamer can be synthesized. Since the SELEX was first introduced a variety of other methods and variations of producing aptamers have been developed. These methods are known to those of skill in the art and are within the scope of this invention.

### Immobilized Binding Agent.

There are a large variety of different support matrixes that can be used. They include beads, membranes and solid surfaces and they are made from different materials such as agarose, and various plastics and acrylics. There are a variety of methods for attaching the binding agent to the support matrix. One method is by passive adsorption. Typically the binding agent is dissolved in water or a coating buffer and the support matrix is immersed in this solution. After a period of time to allow the binding agent to adsorb onto the support matrix the matrix is washed with water or buffer to remove unbound material. After the binding agent is attached to the support matrix any remaining active surfaces can be blocked using a solution of human albumin or similar blocking material. The coated matrix is then stored dry or in a preservative solution. To further ensure stability the apheresis device may be vacuum packed or stored under nitrogen. It may be stored refrigerated or at room temperature.

Another method is the covalent attachment of the binding agent to the support matrix. On surfaces that are aminated or carboxylated, covalent coupling is achieved using bifunctional cross-linkers that couple the amine or carboxyl group on the surface to a functional group, such as an amine or sulfhydryl, on the biomolecule. Selection of the cross-linker will determine the type of covalent bond that will be formed. Functional and covalently reactive groups, such as N-oxysuccinimide, maleimide and hydrazide groups, can also be grafted onto a suitable surface support via a photo-linkable spacer arm resulting in a reactive surface to covalently attach the binding agent. After the binding agent is attached to the support matrix any remaining active surfaces can be blocked using a solution of human albumin or similar blocking material. The coated matrix is then stored dry or in a preservative solution. To further ensure stability the apheresis device may be vacuum packed or stored under nitrogen. It may be stored refrigerated or at room temperature.

Another method for covalent attachment that is used is to treat the support matrix with 3-aminopropyltriethoxysilane (APTES) and to cross-link the binding agent to the APTES functionalized surface using 1-Ethyl-3-(3-dimethylaminopropyl) carbodiimide (EDC). After the binding agent is attached to the support matrix any remaining active surfaces can be blocked using a solution of human albumin or similar blocking material. The coated matrix is then stored dry or in a preservative solution. To further ensure stability the apheresis device may be vacuum packed or stored under nitrogen. It may be stored refrigerated or at room temperature.

Another method is to use a linking agent to attach the binding agent to the support matrix. For example, avidin is covalently attached to the matrix while the binding agent in solution is biotinylated. When the biotinylated binding agent comes into contact with the avidin it will bind to it and in turn will thus become attached to the matrix. The avidin/biotin system has the advantage of increasing the concentration of immobilized binding agent on the support matrix because each avidin molecule is capable of binding to four biotin molecules. It also orients the binding agent so that the active site on the molecule is exposed. After the binding agent is attached to the support matrix any remaining active surfaces can be blocked using a solution of human albumin or similar blocking material. The coated matrix is then stored dry or in a preservative solution. To further ensure stability the apheresis device may be vacuum packed or stored under nitrogen. It may be stored refrigerated or at room temperature.

There are numerous established methods of attaching antibodies, binding peptides and aptamers to a variety of different surfaces. The method selected will depend on the moiety to be attached and the physical and chemical composition of the support matrix. These and other means of attachment will be obvious to one of skill in the art and are therefore considered to be within the spirit and scope of this invention.

In one embodiment of this invention two or more binding agents are combined together to bind out two or more harmful factors from blood. For example, each binding agent is prepared separately (e.g. anti-sFlt-1 antibody coated beads and anti-sEndoglin antibody coated beads) and the two batches of beads are then mixed together within the targeted apheresis device or chamber. Or in the case where a support membrane is used multiple binding agents can be simultaneously attached to the membrane. Depending on the number of different binding agents used multiple different harmful factors can be removed. These include sFlt-1, sEndoglin, Endothelin-1, tumor necrosis factor (TNF) and interleukin-1 (IL-1), interleukin 6 (IL-6) and interleukin 8 (IL-8). At the same time small molecule harmful factors such as digoxin, digitalis-like factor, ouabain, ouabain-like factor, marinobufogenin (MGB), and marinobufotoxin (MBT) are removed using dialysis.

In one embodiment of this invention a method of ultrafiltration is used instead of dialysis in order to remove water along with small molecule harmful factors. Women with pre-eclampsia frequently have edema in their tissues. It has been postulated that an increase in extracellular fluid triggers the production of endogenous Na/K ATPase inhibitor which then inhibits the Na/K ATPase on vascular smooth muscle cells causing vasoconstriction and hypertension. It is logical to assume that reduction in the amount of extracellular fluid will not only reduce strain on the heart and kidneys but will also inhibit the production of endogenous inhibitor and thus prevent the development of hypertension. In order to reduce excess fluid and to remove small molecule harmful factors a differential pressure is applied across the semipermeable membrane that has an exclusion limit of about MW 30,000 or less. This causes water and small molecules including harmful factors such as Na/K ATPase inhibitors to be filtered out and removed and the treated blood returned to the patient.

In one embodiment of this invention the method of dialysis and the method of ultrafiltration are combined to simultaneously dialyse and filter out small molecules from the blood. The procedure is termed “diafiltration” and is known to those of skill in the art.

In this invention a woman with pre-eclampsia will be treated with one or more apheresis sessions depending on the clinical symptoms, blood pressure readings, fetal distress and the results of various monitoring and laboratory tests. We anticipate that the combined targeted apheresis and dialysis and/or filtration procedure described in this invention will ameliorate the symptoms of pre-eclampsia and postpone the decision to induce early delivery of the baby for a certain number of days. If the conditions then worsens the apheresis treatment can be repeated one or more times to further prolong pregnancy until such time as the baby is delivered at term or as close to term as possible.

This invention teaches the removal of harmful factors from whole blood. It will be obvious to those of skill in the art that the same apheresis process disclosed in this invention can be used to remove harmful factors from plasma. Said plasma can be isolated using differential centrifugation or membrane filtration and then processed using targeted apheresis combined with dialysis and/or ultrafiltration. After removal of the harmful factors the cleaned plasma is remixed with the blood cells and returned to the patient. Plasma that is processed according to the teaching in this invention is therefore considered to lie within the spirit and scope of this invention.

This invention discloses a novel means of treating high blood pressure in pre-eclampsia using targeted apheresis combined with dialysis and/or ultrafiltration. It will be obvious to one of skill in the art from the principles disclosed herein, and the materials and methods used, that there are numerous variations and modifications that can be made without departing from the spirit and scope of this invention. Therefore any such changes made are considered to lie within the scope of this invention.

1. A method of removing multiple harmful factors associated with high blood pressure from the blood or plasma of a woman with pre-eclampsia using targeted apheresis combined with dialysis and/or ultrafiltration

2. A method according to claim 1 wherein said method comprises two interconnected devices, wherein one device is a targeted apheresis device that contains immobilized binding agents that can target and remove harmful factors that are proteins; and a second device containing a semipermeable membrane that can dialyze and/or filter out small molecule harmful factors.

3. A method according to claim 1 wherein said method comprises a single device divided into two interconnected compartments, wherein one compartment contains immobilized binding agents and is used to target and remove harmful factors that are proteins; and a second compartment containing a semipermeable membrane that can dialyze and/or filter out small molecule harmful factors.

4. A method according to claim 1 wherein said method comprises a single device containing a semipermeable membrane coated with the binding agents; wherein the harmful factors that are proteins are targeted and bound out; and simultaneously the small molecule harmful factors are dialyzed and/or filtered out.

5. A method according to claim 1 wherein the harmful factors to be removed include two or more of the following: sFlt-1, sEndoglin, Endothelin-1, digoxin, digitalis-like factors, ouabain, ouabain-like factors, marinobufogenin, marinobufotoxin, and optionally one or more pro-inflammatory cytokines including Tumor Necrosis Factor, Interleukin-1, Interleukin-6, and Interleukin-8.

6. A method according to claim 1 wherein the method of targeted apheresis utilizes immobilized binding agents that are antibodies, or aptamers, or binding peptides; and wherein said binding agents target and remove the specific harmful factors associated with causing high blood pressure in women with pre-eclampsia.

7. A method according to claim 6 wherein the binding agents are immobilized on a support matrix consisting of:
(a) beads such as agarose beads, or plastic beads, or latex beads, or
(b) granular plastic sheets or
(c) porous support membranes, or
(d) a semipermeable membrane.

8. A method according to claim 1 wherein the woman with pre-eclampsia will receive one or more treatments of targeted apheresis combined with dialysis and/or ultrafiltration in order to reduce the symptoms of the disease and extend the duration of pregnancy to term or as close to term as possible.
